(19) United States
(12) Patent Application Publication
(10) Pub. No.: US 2023/0270582 A1
(43) Pub. Date: Aug. 31, 2023

(54) A male urinary incontinence device

(71) Applicant: Coloplast A/S, Humlebaek (DK)

(72) Inventors: Kim Becker, Hilleroed (DK); Kristoffer Zeuthen, Copenhagen OE (DK); Rune Stagstrup, Broendby Strand (DK); Chaban Bougherara, Frederiksberg C (DK); Marie Bay Borg, Broenshoej (DK)

(21) Appl. No.: 18/016,371

(22) PCT Filed: Jul. 15, 2021
(86) PCT No.: PCT/DK2021/050242

§ 371 Date: Jan. 16, 2023

(30) Foreign Application Priority Data
Jul. 17, 2020 (DK) PA 2020 70496

(51) Int. Cl. A61F 5/453

(52) U.S. Cl. CPC A61F 5/453

(57) ABSTRACT

A male urinary incontinence device (10) comprising a proximal wall (20) and a distal wall (30) being sealed together at the periphery to form an inner cavity (70), the proximal wall comprises a through-going inlet opening (25) through which a penis can be entered, the inner cavity adapted to contain at least a part of the penis, and wherein the distal wall comprises a second opening (35) configured for allowing access to the inner cavity, the through-going inlet opening comprises a circular shape, and where the through-going inlet opening (25) is positioned symmetrically at a longitudinal central axis X defined by a line of symmetry in radial direction of the device.

The invention relates to a male urinary incontinence device.

### BRIEF DESCRIPTION OF THE DRAWING

The accompanying drawings are included to provide a further understanding of embodiments and are incorporated into and a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 is an isometric exploded view of a male urinary incontinence device,

FIG. 2 is a proximal side view of a male urinary incontinence device,

FIG. 3 is a distal side view of a male urinary incontinence device,

FIG. 4 is a distal side view of a male urinary incontinence device,

FIG. 5 is an enlarged partial side view of a male urinary incontinence device,

FIG. 6 is a side view of a male urinary incontinence device,

FIG. 7 is a side view showing attachment of the absorber element,

FIG. 8 is a side view and a cross-sectional view in longitudinal direction of a male urinary incontinence device,

FIG. 9 is an isometric exploded view of a male urinary incontinence device.

### DETAILED DESCRIPTION

Embodiments, and features of the various exemplary embodiments described in this application, may be combined with each other (“mixed and matched”), unless specifically noted otherwise.

In the following detailed description, reference is made to the accompanying drawings, which form a part hereof, and in which is shown by way of illustration specific embodiments in which the invention may be practiced. Because components of embodiments can be positioned in different orientations, the directional terminology is used for purposes of illustration and is in no way limiting. It is to be understood that other embodiments may be utilized and structural or logical changes may be made without departing from the scope of the present invention. Therefore, the following detailed description is not to be taken in a limiting sense, and the scope of the present invention is defined by the appended claims.

Throughout this disclosure, the words “patient” or “user” are used to address the person to wear the male urinary incontinence device. However, in some cases “user” may also relate or refer to a health care professional (HCP), such as a surgeon or continence care nurse or others. In those cases, it will either be explicitly stated, or be implicit from the context that the “user” is not the “patient” him- or herself.

In the following, whenever referring to a proximal side of a device or part of a device, the proximal side is the side closest to the user, when the device is fitted on a user and the distal side is the opposite side—the side furthest away from the user in use.

The axial direction is defined as the direction of the penis when the device is worn by a user.

The radial direction is defined as transverse to the axial direction that is transversely to the direction of the penis. Thus, the radial direction is substantially perpendicular to the penis of the user.

The longitudinal central axis X is defined by a line of symmetry in radial direction of the male urinary incontinence device. The longitudinal central axis X is substantially equal to an imaginary vertical line of gravity when the device is worn by a user in standing position.

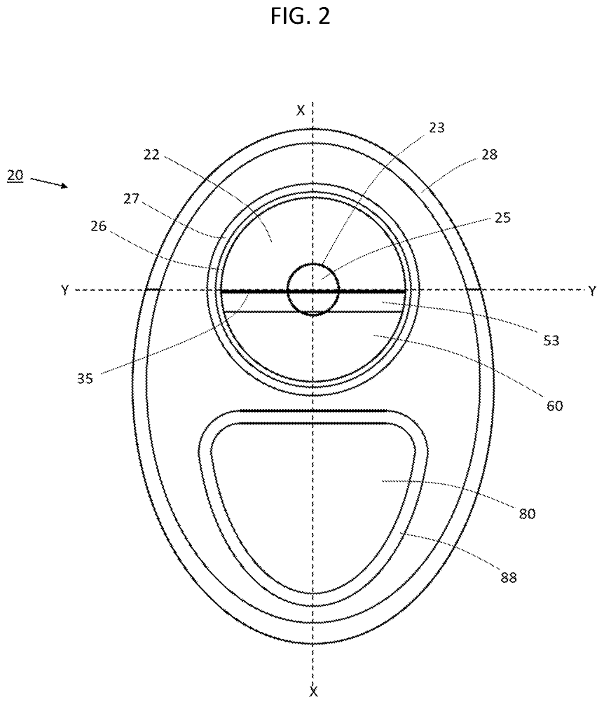

The transverse axis Y extends perpendicular to the longitudinal central axis X through the centre of an inlet opening in radial direction. Both the longitudinal central axis X and the transverse axis Y extends in radial direction, se FIG. 2.

The longitudinal direction is the direction along the longitudinal central axis X. The transverse direction is the direction perpendicular to the longitudinal direction, which corresponds to the direction along the transverse axis Y extending transverse the male urinary incontinence device.

In some sentences, the words “inner” and “outer” may be used. These qualifiers should generally be perceived with respect to the radial or axial direction, such that a reference to an “outer” element means that the element is farther away from a centre portion of the incontinence device than an element referenced as “inner.”

Generally, the male urinary incontinence device is symmetrically shaped along the longitudinal central axis X. The transverse axis may be arranged transverse the male urinary incontinence device at the widest portion of the device.

The use of the phrase “substantially” as a qualifier of certain features or effects throughout this disclosure, is intended to simply mean that any deviations are within tolerances that would normally be expected by the skilled person in the relevant field.

Many people suffer from light incontinence. Incontinence may occur in a variety of ways. Stress incontinence means that you leak urine when you sneeze, cough, laugh, lift something, change position, or do something that puts stress or strain on your bladder. Urge incontinence is an urge to urinate that is so strong that you cannot make it to the toilet in time. It also happens when your bladder squeezes when it should not. This can happen even when you have only a small amount of urine in your bladder. Overactive bladder is a kind of urge incontinence. Where the amount of leaked urine in stress incontinence may be quite small such as few drops, the amount of leaked urine in urge incontinence may be larger, such as up to 180 ml a day.

Male urinary incontinence aiding systems are known. Widely used systems known as external urinary catheters and urisheaths normally comprise a roll-on sheath or body portion for enclosing the shaft of the penis, and a tip portion that is provided with a comparatively short discharge tube, which via a tube is connected to a urine collection bag that is fastened to the leg of the user. The sheath portion is rolled-up in a number of successive windings to such an extent that adhesive on the inner surface of the sheath is accommodated in the windings. However, unrolling of a sheath correctly on a penis can be very challenging and it is essential that the sheath is fitted correctly to ensure a good sealing between the skin of the penis and the sheath. The known systems are especially intended and designed for persons suffering from medium to high urine incontinence, and the urine collection bags having a capacity of 500 ml are rather big and inconvenient to wear.

There is a need for a lightweight and discreet male urinary incontinence device that may conveniently be sealingly arranged on the penis and having a smaller capacity of about 100-300 ml and be more agreeable for the user to wear in case of lighter incontinence.

Incontinent users and health care professionals alike would welcome improvements in continence care devices to better meet such requirements.

Embodiments relate to a male urinary incontinence device comprising a proximal wall and a distal wall being sealed together at the periphery to form an inner cavity, the proximal wall comprises a through-going inlet opening through which a penis can be entered, the inner cavity adapted to contain at least a part of the penis, and wherein the distal wall comprises a second opening configured for allowing access to the inner cavity, the through-going inlet opening is positioned symmetrically at a longitudinal central axis X defined by a line of symmetry in radial direction of the device and the second opening is positioned opposed the through-going inlet opening in axial direction and wherein the through-going inlet opening and the second opening are coincidently aligned along the longitudinal central axis X and the through-going inlet opening and the second opening are positioned coincidently aligned along a transverse axis Y extending perpendicular to the longitudinal central axis X in radial direction.

Thus, when the second opening is closed, the device defines an enclosure for the penis.

When the second opening is open, it facilitates easy application of the device to the penis as both sides of the aperture are accessible and the penis can be pulled through the inlet opening and into the inner cavity. Furthermore, by opening the second opening, the user can retrieve the penis from the device, having the penis penetrating both openings, without detaching the device, and urinate naturally. In embodiments, where the device is provided with an absorbent element, this can be inspected or exchanged through the second opening.

The proximal wall and the distal wall may be welded together at the periphery. The through-going inlet opening may comprise a circular shape. The through-going inlet opening is positioned at a distance from the periphery of the proximal wall of male urinary incontinence device. The second opening may be liquid-tight and/or splash-proof closable.

The second opening may be adapted to be opened and closed several times, e.g. such as 10 times before the device may be disposed.

In embodiments, the through-going inlet opening comprises a circular shape configured to fit around the shaft of a penis.

The through-going inlet opening is positioned symmetrically at a longitudinal central axis X defined by a line of symmetry in radial direction of the device.

Generally, the periphery of the device may comprise a symmetrical shape having one line of symmetry in radial direction, such as a pear-shaped periphery. The periphery of the device may comprise an outer shape having at least two lines of symmetry, such as an oval-shaped periphery. The periphery may comprise other suitable geometric forms.

The second opening is positioned opposed the through-going inlet opening in axial direction and the through-going inlet opening and the second opening are coincidently aligned along the longitudinal central axis X.

The through-going inlet opening, and the second opening are positioned coincidently aligned along a transverse axis Y extending perpendicular to the longitudinal central axis X in radial direction.

In another embodiment, the second opening may be located staggered in longitudinal direction off-set the transverse axis Y.

In embodiments, the distal wall comprises an upper wall portion and a lower wall portion, the upper wall portion comprising a first edge and the lower wall portion comprising a second edge positioned juxtaposed the first edge, the first edge and the second edge defines the periphery of the second opening.

In embodiments, the second opening comprises an elongated slit-shape extending in a direction parallel to a transverse axis Y.

Hereby, is achieved a more cost-effective manufacturing of the device, as the second opening are aligned in parallel with the direction of a conveyer in production lines, thus the device is easier to produce.

In embodiments, the distal wall comprises an upper wall portion and a lower wall portion, the upper wall portion comprising a first edge and the lower wall portion comprising a second edge juxtaposed the first edge, the first edge and the second edge defines the periphery of the second opening.

In embodiments, the first edge of the upper wall portion of the distal wall comprises a hinged flap portion adapted to cover the second opening.

In embodiments, the hinged flap portion comprising an adhesive strip layer at a proximal surface of the hinged flap portion adapted to adhere to the second edge of the lower wall portion of the distal wall. In embodiments, the second edge comprises a closure film layer adapted for forming a re-closable opening with the adhesive strip layer.

In embodiments, the hinged flap portion comprises a distal surface comprising tactile structure for providing tactile guidance for access to the second opening.

In embodiments, a distal surface of the hinged flap portion comprises a color for visually illustrating the flap for providing guidance to access the second opening.

In embodiments, the hinged flap portion comprises a reinforced foam layer. The foam layer may comprise a size in radial direction of 15-27 mm, such as 21 mm.

The outer surface on the distal side of the hinged flap portion may comprise a rough surface in forms of small nubs to provide tactile guidance to a user and facilitate the user to find the second opening covered by the hinged flap portion.

In embodiments, the reinforced foam layer protrudes further than the adhesive strip layer in longitudinal direction to form a non-adhesive grip portion.

Generally, the proximal wall and the distal wall comprises a welding zone along the periphery of the proximal wall and the distal wall. The proximal wall and the distal wall may be welded together at the periphery.

Generally, the male urinary incontinence device may comprise a length of 160-210 mm, such as 180-190 mm along the longitudinal central axis X and a width of 90-140 mm, such as 110-120 mm along the transverse axis Y.

In embodiments, the upper wall portion comprises a radially inwardly extending sealing zone for reinforcement of the first edge adjacent along the edge of the second opening.

In embodiments, the proximal wall comprises an inlet opening rim and the through-going inlet opening formed by a flexible cuff member, the flexible cuff member may be adapted to increase in a size corresponding to the size of the inlet opening rim.

The flexible cuff member may be composed of elastic and/or flexible material, such as silicone. The flexible cuff member may be attached to the inlet opening rim at a cuff member welding zone. The flexible cuff member may comprise a membrane and a tubular shaped collar portion, which defines the through-going inlet opening. The proximal wall and the membrane may extend substantially parallel in a first plane, in axial direction, and the tubular shaped collar portion may extend transverse to the first plane, in radial direction. The cuff member may be so flexible that the through-going inlet opening may obtain approx. same size as the diameter of the inlet opening rim.

The flexible cuff member comprising the membrane and the tubular shaped collar portion are configured to allow elastic expansion of the aperture to fittingly engage with the penis.

The flexible cuff member may be flexible to an extend that the size of the through-going inlet opening may increase to a size, during mount of the device, corresponding to the size of the inlet opening of the proximal wall.

The flexible cuff member may be manipulated and stretched to similar size of the inlet opening rim allowing the user via the second opening to mount the flexible cuff member on the shaft of a penis. After mount of the device at least the tip of a penis is in the inner cavity juxtaposed in-between the proximal wall and the distal wall.

The proximal wall and a membrane of a cuff member may extend substantially parallel in a first plane in radial direction and the tubular shaped collar portion of the flexible cuff member may extend transverse to the first plane.

The flexible cuff member may be formed by flexible material and be very flexible and stretchable particularly in radial direction for easier mount of the male urinary incontinence device.

In embodiments, the cuff member welding zone surrounding the inlet opening rim comprise a circular shape opening having a diameter of 65-90 mm, such as 70-76 mm. The cuff member comprises a circular shape opening having a diameter approx. 4-8 mm larger than the size of the cuff member welding zone. The proximal wall comprises a circular shaped opening having a diameter approx. 4-8 mm smaller than the size of the cuff member welding zone.

In embodiments, the proximal wall is a layered sheet comprising of a textile sheet and e film sheet. A punching contour of the layered sheet comprises a size of 66 mm, defining the inlet opening of the proximal wall. The cuff member comprises a diameter of 80 mm, the cuff member and the inlet opening overlaps at the cuff member welding zone comprising a diameter of 70-76 mm.

In embodiments, the male urinary incontinence device comprises an absorber element comprising absorbent material, the absorber element being positioned in the inner cavity.

In embodiments, the absorber element is detachable attached to the inner surface of the lower wall portion of the distal wall.

The distal wall comprises a first edge and a second edge formed by the upper and lower wall portions, which defines the shaped of the second opening.

Generally, the absorber element may be attached to the lower wall portion of the distal wall by welding at an absorber support weld positioned in a distance from the second opening of 5-40 mm or less, such as 10-25 mm, 16-19 mm or less from the second edge of the lower wall portion. The absorber support weld may extend in a direction parallel with transverse axis Y in a distance d from the welding zones of the periphery of the distal wall.

A small gap between the two welding zones of the upper wall portion and the lower wall portion of the distal wall may be provided.

The welding zone along the upper wall portion 40 may comprise a radial inwardly extending additional welding zone providing additional support for the hinged flap portion to avoid separation along the edge of the second opening and the hinged flap portion.

In embodiments, the absorber element comprises the size that are at least 5.5 mm, such as 9.5 mm smaller than the inner surface of the welding zone of the lower wall portion. Hereby is provided a distance d in the inner cavity between the absorber element and the welding zone at the periphery, enable the absorber element to be moved relative to the walls of the device.

In embodiments, the device comprises a comfort patch on the inner surface of the proximal wall. The comfort patch may be positioned opposed the absorber element adjacent the distal wall. The comfort patch may be formed by a textile sheet layer.

In embodiments, the comfort patch is positioned symmetrically aligned the longitudinal central axis X adjacent the rim of the inlet opening extending toward the lower end of the device in radial direction. The comfort patch may be radially displaced from the inlet opening rim alone the longitudinal central axis X.

The use of a male urinary incontinence device comprises the steps of; expanding the through-going inlet opening of the flexible cuff member, positioning the through-going inlet opening of the flexible cuff member around the penis by assessing the through-going inlet opening of the flexible cuff member from proximal side and through the second opening from the distal, closing the second opening by adhering the hinged flap portion the second edge of the distal wall. The use may further comprise the step of opening the hinged flap portion to allow user to urinate or inspect the absorber element and/or the inner cavity.

### DETAILED DESCRIPTION OF THE DRAWING

The accompanying drawings are included to provide a further understanding of embodiments and are incorporated into and a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

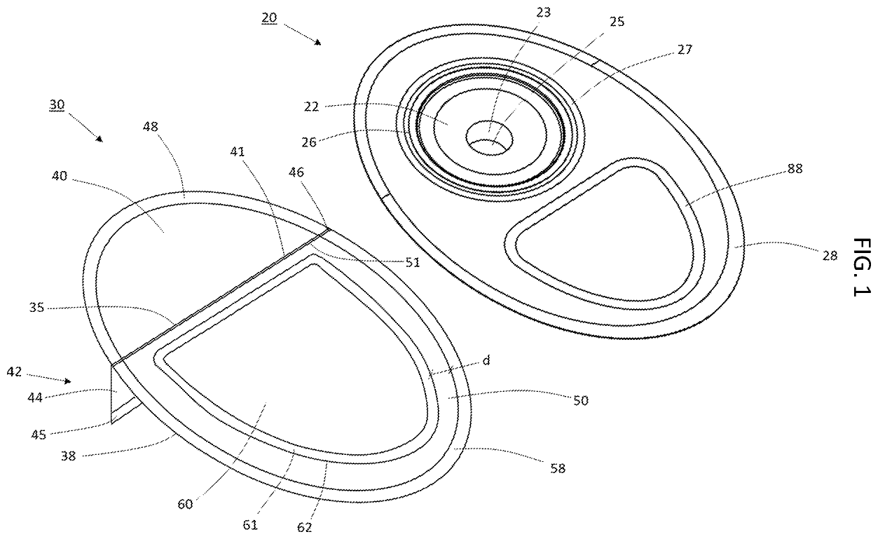

In FIGS. 1-9 is shown a male urinary incontinence device from different views.

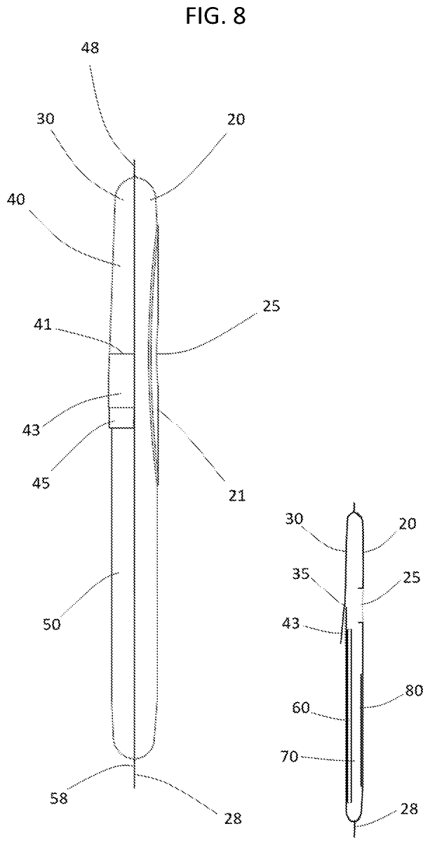

FIG. 1 illustrates the male urinary incontinence device comprising a proximal wall 20 and a distal wall 30. The periphery 28 of the proximal wall 20 and the periphery 38 of the distal wall are sealed together to form a pouch with an inner cavity 70 (as illustrated in FIG. 8). The proximal wall 20 and the distal wall 30 are welded together at the periphery.

The proximal wall 20 comprises a through-going inlet opening 25 through which a penis can be entered.

The distal wall 30 is divided into two wall portions, an upper wall portion 40 and a lower wall portion 50. The upper wall portion 40 comprising a first edge 41 and the lower wall portion 50 comprising a second edge 51 positioned juxtaposed the first edge 41. The first edge 41 and the second edge 51 of the distal wall 30 defines the periphery of the second opening 35. The second opening 35 comprises a slit-shaped form.

The upper wall portion 40 comprises a welding zone 48 and the lower wall portion 50 comprises a welding zone 58. The welding zones 48, 58 extends along the periphery 38 of the distal wall 30.

The upper wall portion 40 comprises a hinged flap portion 42. The hinged flap portion 42 overlaps the lower wall portion 50 and closes the second opening 35 formed between the upper and lower wall portions 40,50 of the distal wall 30. On the inner surface of the hinged flap portion 42 an adhesive strip layer 44 is configured to adhere to a closure film layer positioned on the second edge 51 on the lower wall portion 50 of the distal wall 30, such that the second opening is re-closeable by the adhesive strip layer being in contact with the closure film layer. The hinged flap portion 42 comprises a reinforced foam layer forming a grip portion 45.

The proximal wall 20 comprises an inlet opening rim 26. A flexible cuff member 21 is composed of elastic and/or flexible material, such as silicone. The flexible cuff member 21 is attached to the inlet opening rim 26 at a cuff member welding zone 27. The flexible cuff member 21 comprises a membrane 22 and a tubular shaped collar portion 23, which defines the through-going inlet opening 25. The proximal wall 20 and the membrane 22 extend substantially parallel in a first plane, in axial direction, and the tubular shaped collar portion 23 extends transverse to the first plane, in radial direction. The cuff member 21 may be so flexible that the through-going inlet opening 25 may obtain approx. same size as the diameter of the inlet opening rim 26.

A user may get access to the flexible cuff member 21 and the through-going inlet opening 25 through the second opening oppositely positioned the male urinary incontinence device to manipulate the size and shape of the through-going inlet opening 25 and to allow positioning of the device into position on the penis. When the tubular shaped collar portion 23 is in position, the through-going opening 25 surrounds the penis in a liquid tight manner. Thereafter the second opening may be closed by the hinged flap portion 42.

The proximal wall 20 comprises a nonwoven welding zone 88 for a comfort patch positioned at the distal surface of the proximal wall 20, to provide comfort for the user, when the device is in use.

The male urinary incontinence device comprises an absorber element 60 having a welding zone 61. The absorber element 60 is positioned adjacent the second opening 35 of the distal wall 30. The distance d between the edge 62 of the absorber element 60 and the welding zone 58 of the lower wall portion 50 is 2-4 mm or larger allowing the absorber element to increase in size when absorbing liquid, such as urine.

The absorber element 60 may comprise a heat weldable PE layer a core of absorber material and a non-women material, the PE layer and the non-women material being welded at the welding zone 61 having a width of approximately 5-6 mm and a core absorber area of approximately 6200 mm².

FIG. 2 illustrates the male urinary incontinence device in a side view showing the proximal wall 20.

The proximal wall 20 comprises an inlet opening rim 26. A flexible cuff member 21 comprising of elastic and/or flexible material, such as silicone being attached to the inlet opening rim 26 at a flexible cuff member welding zone 27. The flexible cuff member comprises a membrane 22 and a tubular collar portion 23, which defines the through-going inlet opening 25.

The proximal wall 20 and the membrane 22 extend substantially parallel in a first plane and the tubular shaped collar portion 23 extends transverse to the first plane. The flexible cuff member is made from see-through silicone and the second opening 35 and the absorber element 60 is visible through the flexible cuff member 21. The membrane 22 may comprise varying thickness, such that the thickness increases radially towards the inlet opening rim 26.

FIG. 2 illustrates a longitudinal central axis X extending alone the line of symmetry in radial direction of the device radially through the center of the male urinary incontinence device in longitudinal direction of the device. A transverse axis Y extends in radial direction, through the centre of the through-going inlet opening 25, perpendicular to the longitudinal central axis X.

The through-going inlet opening 25 comprises a circular shape. The longitudinal central axis X and the transverse axis Y intersects each other at the midpoint of the through-going inlet opening 25.

The second opening 35 comprises an elongated slit-shape extending parallel to a transverse axis Y and the second opening is located opposed the through-going inlet opening 25. The absorber element 60 is located in the inner cavity adjacent the second opening 35 below the transverse axis Y.

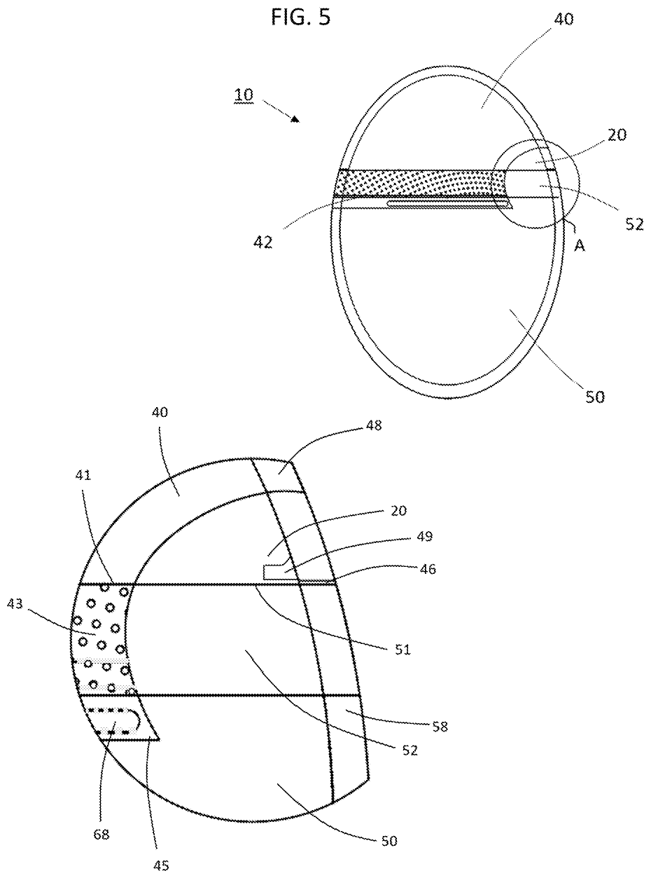

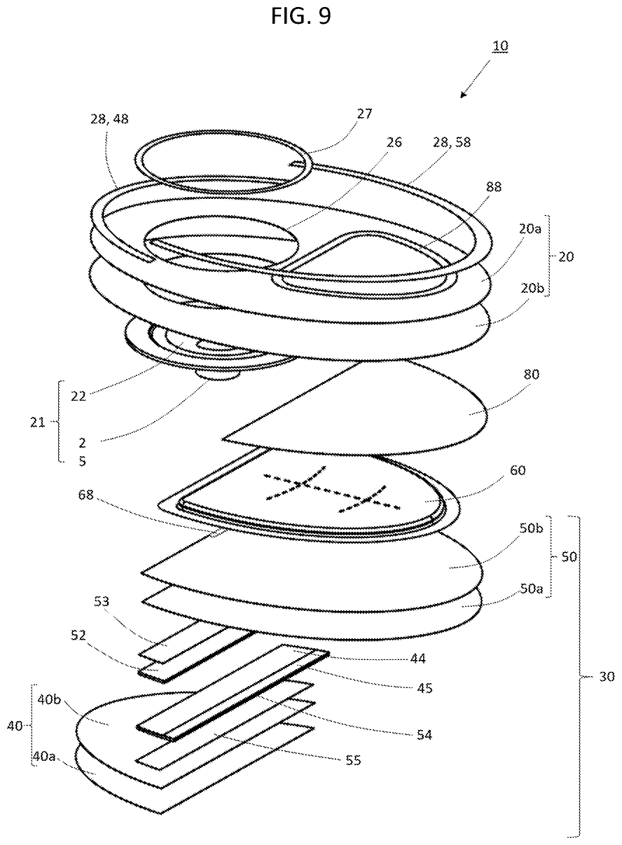

Between the second opening 35 and the absorber element 60 the distal wall comprises a welding zone 53 for a closure film configured to functioning as contact surface for a hinged flap portion comprising adhesive (shown in FIGS. 5 and 9).

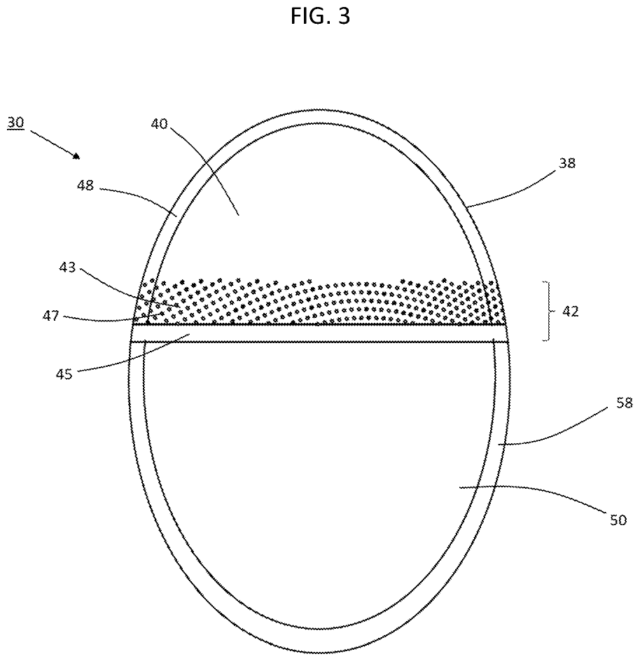

FIGS. 3, 4 and 5 illustrates the male urinary incontinence device 10 in a side view showing the distal wall 30 and an enlarged detail A thereof. The distal wall 30 comprises the upper wall portion 40 and a lower wall portion 50 having a welding zone 48,58 at the periphery 38 of the distal wall 30. The upper wall portion 40 comprises a hinged flap portion 42. The hinged flap portion 42 overlaps the lower wall portion 50 and closes the second opening formed between the upper and lower wall portions 40,50.

The first edge 41 of the upper wall portion 40 is provided with a hinged flap portion 42 adapted to cover the second opening 35 and allowing the second opening 35 to be opened and closed by a user.

The outer surface of the hinged flap portion 42 on the distal surface 43 comprises a tactile structure 47 in forms of small nubs or the like providing tactile guidance to a user to find the second opening 35 covered by the hinged flap portion 42. Furthermore, the hinged flap portion 42 comprises a layer of reinforced foam layer forming a protruding grip portion 45.

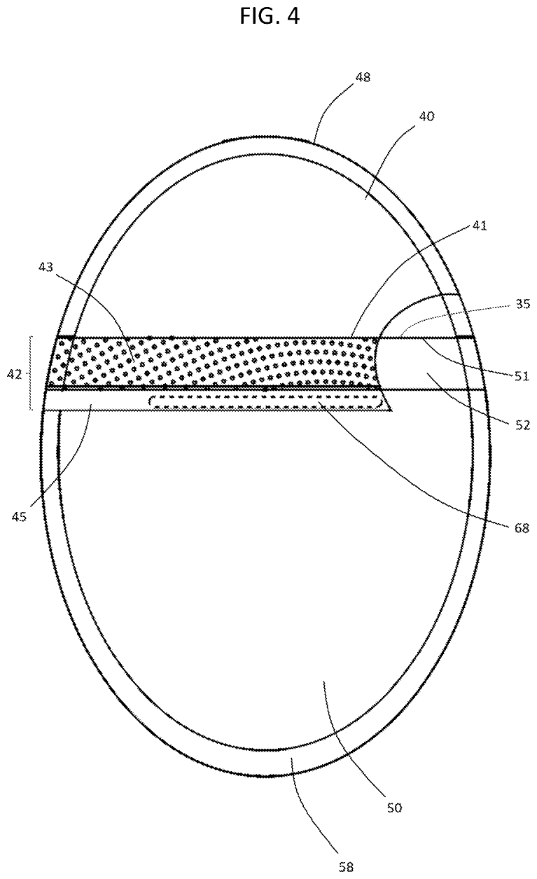

The second opening 35, formed by the first edge 41 and the second edge 51, is closed by the hinged flap portion 42. The absorber support weld 68 illustrated by a dotted line, in FIG. 4, is cover by the hinged flap portion 42.

The hinged flap portion 42 comprises a distal surface comprising tactile structure and a reinforced foam layer 54. The distal surface of the hinged flap portion 42 may comprise a color for visually illustrate the flap and access to the second opening 35.

FIG. 5 illustrates an enlarged detail A of the device. The periphery of the distal wall 35 comprises a welding zone 48 along the upper wall portion 40 and a welding zone 58 along the lower wall portion 50. A small gap 46 between the two welding zones 48, 58 is provided. The welding zone along the upper wall portion 40 comprises an additional radial inwardly extending welding zone 49.

The second edge 51 comprises a closure film layer 52 adapted for forming a re-closable opening with the adhesive strip layer 44. The hinged flap portion 42 comprises an outer flange comprising a foam layer for allow better grip without adhesive.

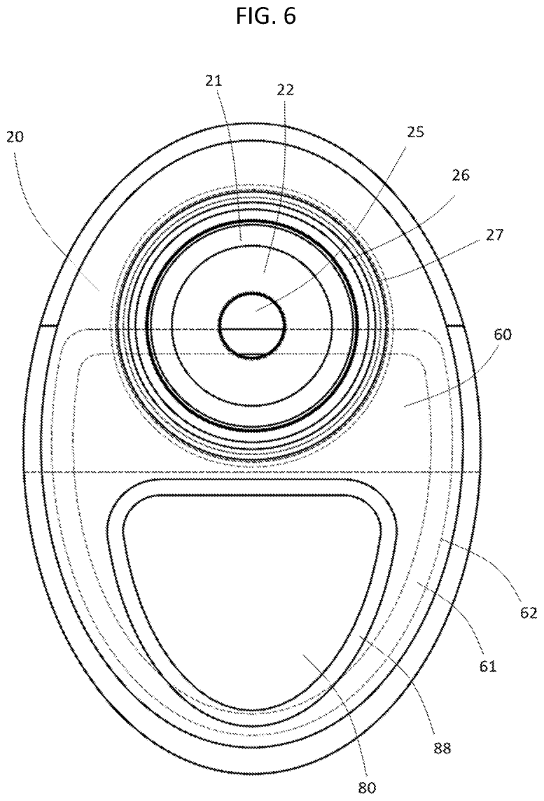

FIG. 6 illustrates the position of the welding zones 61,88 for the absorber element 60 and the comfort patch 80.

The absorber element 60 is located at the proximal surface of the distal wall 30 and the comfort patch 80 is located at distal surface of the proximal wall 20.

The cuff member welding zone 27 surrounding the flexible cuff member 21 and the inlet opening rim 26 of the proximal wall. The cuff member welding zone 27 and the inlet opening rim 26 comprise a circular shape and they are concentric positioned. The diameter of the cuff member welding zone may be approximal 70-76 mm.

The flexible cuff member 21 may be manipulated and stretched to similar size of the inlet opening rim 26 allowing the user via the second opening to mount the flexible cuff member 21 on the shaft of a penis. After mount of the device at least the tip of a penis is in the inner cavity juxtaposed in-between the comfort patch and the absorber element.

The size of the through-going inlet opening 25 may be manipulated in size by the user to increase the diameter to a size corresponding to the size of the inlet opening rim 26 of the proximal wall 20.

In an embodiment, the proximal wall 20 is a layered sheet comprising of a textile sheet and e film sheet (as illustrated in FIG. 9). A punching contour of the layered sheet comprises a size of 66 mm, defining the inlet opening 26 of the proximal wall 20. The cuff member 21 comprises a diameter of 80 mm, the cuff member 21 and the inlet opening 26 overlaps at the cuff member welding zone 27 comprising a diameter of 70-76 mm.

The comfort patch 80 is positioned adjacent the inlet opening rim 26 of the proximal wall and may overlap in axial direction with the absorber along the outer periphery of the device.

The inlet opening 25 and the second opening 35 is located opposed each other in radial direction, thus due to the form and size of the two openings 25,35 the extent of the comfort patch in longitudinal direction along the longitudinal central axis X is smaller than the absorber element 60.

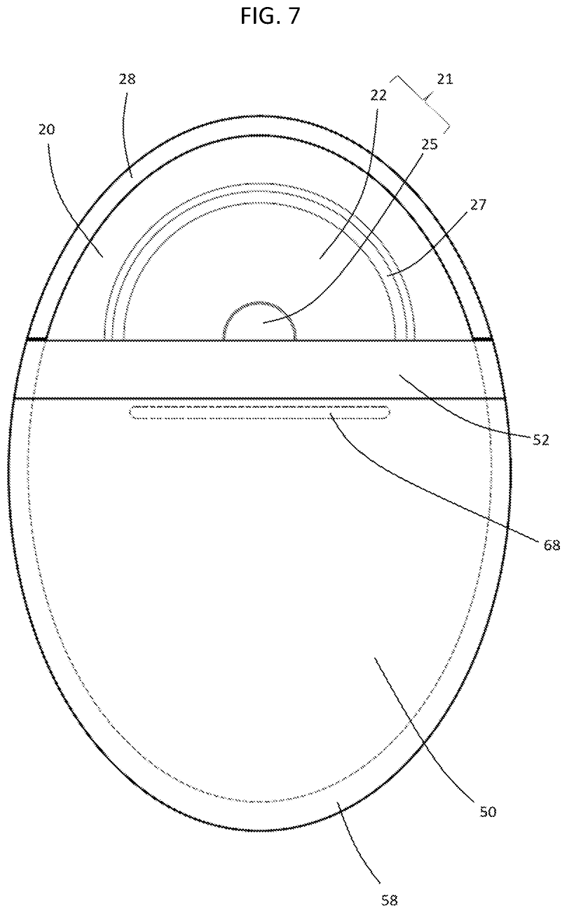

FIG. 7 illustrates that the second edge of the lower wall portion 50 comprises a closure film layer 52 adapted for forming a re-closable opening with the adhesive strip layer of the hinged flap portion.

The absorber support weld 68 is positioned below the closure film layer 52.

The flexible cuff member 21 comprises the membrane 22 and the through-going inlet opening 25. The flexible cuff member 21 is surrounded by the cuff member welding zone 27.

FIG. 8 illustrates a side view and a cross sectional side view of the male urinary incontinence device comprising a proximal wall 20 and distal wall 30 joint at the periphery by welding. The proximal wall 20 and the distal walls 30 encloses a volume defining the inner cavity 70. The comfort patch 80 is attached to the inner surface of the proximal wall 20 and the absorber element 60 is attached to the distal wall 30. In radial direction, the comfort patch 80 and the absorber element 60 is positioned opposed each other as well as the through-going inlet opening 25 and the second opening 35. The second opening 35 is closed off by the hinged flap portion 42, which is hingedly attached to the upper wall portion 40 at the first edge 41.

FIG. 9 illustrates an exploded isometric view of the male urinary incontinence device 10.

The device comprises the proximal wall 20 and the distal wall 30, each wall consists of a textile sheet 20a,40a,50a and a film sheet 20b,40b,50b. The male urinary incontinence device comprises sheets and layers forming a device having a compact outer shape when not in use.

The film sheets 20b,40b,50b is provided on the inner surface of the textile sheets of the walls as to provide a liquid tight enclosure.

The different portion of the device are welded together at welding zones 27,28,48,58,88,68,53. The absorber element 60 is welded to the second edge of the lower wall portion 50.

After mount of the device at least the tip of a penis is in the inner cavity juxtaposed in-between the comfort patch attached to the proximal wall and the absorber element attached to the distal wall.

The first and second edge 41,51 of the upper wall portion 40 and the lower wall portion 50 comprises complementary closing means, in the form of an adhesive strip layer 44 and a closure film layer 52. The complementary closing means allows a user to open and close the device several times. The closure film layer 52 configured to functioning as contact surface for a hinged flap portion comprising adhesive

The upper wall portion 40 comprises a reinforced foam layer 54 attached by welding illustrated by a welding zone 55. The foam layer 54 comprises an adhesive strip layer 44 adapted to adhere to the closure film layer 52. The protrusion of the reinforced foam layer 54 extends beyond the adhesive strip layer 44 to form the adhesive-free grip portion 45.

1-18. (canceled)

19. A male urinary incontinence device comprising:
a pouch including a proximal wall sealed along an entirety of an outer periphery of the proximal wall to a distal wall to form an inner cavity inside of the pouch;
an inlet opening formed in the proximal wall, with the inlet opening communicating with the inner cavity of the pouch and sized to receive a penis;
a second opening formed in the distal wall, with the second opening communicating with the inner cavity of the pouch; and
a urine absorbing element disposed inside of the inner cavity of the pouch;
wherein the second opening is positioned opposite of the inlet opening and is adapted to allow a user of the male urinary incontinence device to direct a position of the penis through the inlet opening and into the inner cavity of the pouch.

20. The male urinary incontinence device of claim 19, wherein the inlet opening is positioned on a central longitudinal axis of the proximal wall with lateral symmetry relative to the proximal wall on each lateral side of the inlet opening.

21. The male urinary incontinence device of claim 19, wherein the second opening is formed as an elongated slit extending laterally across a width of the distal wall.

22. The male urinary incontinence device of claim 19, wherein the second opening extends laterally across a width of the distal wall and intersects with a center of the inlet opening.

23. The male urinary incontinence device of claim 19, wherein the inlet opening is circular.

24. The male urinary incontinence device of claim 19, wherein the distal wall comprises an upper wall portion and a lower wall portion, the upper wall portion comprising a first edge and the lower wall portion comprising a second edge, and the first edge and the second edge combine to define a periphery of the second opening.

25. The male urinary incontinence device of claim 19, wherein the distal wall comprises an upper wall portion and a lower wall portion with the second opening positioned between the upper wall portion and the lower wall portion, and the upper wall portion comprises a flap adapted to cover the second opening.

26. The male urinary incontinence device of claim 19, wherein the second opening formed in the distal wall is located between an upper wall portion and a lower wall portion of the distal wall, and the upper wall portion comprises a hinged flap provided with adhesive for attachment to the lower wall portion.

27. The male urinary incontinence device of claim 19, wherein the second opening formed in the distal wall is located between an upper wall portion and a lower wall portion of the distal wall, with the male urinary incontinence device further comprising:
a flap secured to the upper wall portion, with the flap adapted to close the second opening;
adhesive disposed on the flap; and
a closure film disposed on the lower wall portion;
wherein the closure film and the adhesive combine to allow the flap to re-closably seal the second opening.

28. The male urinary incontinence device of claim 19, wherein the second opening formed in the distal wall is located between an upper wall portion and a lower wall portion of the distal wall, with the male urinary incontinence device further comprising:
a flap secured to the upper wall portion, with the flap adapted to close the second opening;
wherein the flap includes a tactile structure adapted to provide the user with tactile guidance in locating the second opening.

29. The male urinary incontinence device of claim 19, wherein the second opening formed in the distal wall is located between an upper wall portion and a lower wall portion of the distal wall, with the male urinary incontinence device further comprising:
a flap secured to the upper wall portion, with the flap adapted to close the second opening;
wherein the flap includes a colored pattern adapted to visually guide the user in locating the second opening.

30. The male urinary incontinence device of claim 19, wherein the second opening formed in the distal wall is located between an upper wall portion and a lower wall portion of the distal wall, with the male urinary incontinence device further comprising:
a flap secured to the upper wall portion, with the flap adapted to close the second opening;
wherein the flap includes a reinforced foam layer.

31. The male urinary incontinence device of claim 19, wherein the second opening formed in the distal wall is located between an upper wall portion and a lower wall portion of the distal wall, with the male urinary incontinence device further comprising:
a flap secured to the upper wall portion, with the flap adapted to close the second opening;
adhesive disposed on the flap; and
a reinforced foam layer disposed on the flap and extending longitudinally past the adhesive to provide the flap with a non-adhesive protruding grip portion.

32. The male urinary incontinence device of claim 19, wherein the proximal wall comprises an elastic cuff formed of silicone, and the inlet opening is formed in the elastic cuff and adapted to adjust in shape and size to accommodate a range of sizes of the penis.

33. The male urinary incontinence device of claim 19, wherein the urine absorbing element is connected to a proximal surface of the distal wall.

34. The male urinary incontinence device of claim 19, wherein the urine absorbing element is detachably secured inside of the pouch.

35. The male urinary incontinence device of claim 19, further comprising:
a textile comfort patch disposed on an inner surface of the proximal wall.
